SEQUENCE LISTING (37 sequences)

SEQ ID NO: 1 - protein, 503 aa, Homo sapiens
MTLGSPRKGLLMLLMALVTQGDPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLALILGPVLALLALVALGVLGLWHVRRRQEKQRGLHSELGESSLILKASEQGDSMLGDLLDSDCTTGSGSGLPFLVQRTVARQVALVECVGKGRYGEVWRGLWHGESVAVKIFSSRDEQSWFRETEIYNTVLLRHDNILGFIASDMTSRNSSTQLWLITHYHEHGSLYDFLQRQTLEPHLALRLAVSAACGLAHLHVEIFGTQGKPAIAHRDFKSRNVLVKSNLQCCIADLGLAVMHSQGSDYLDIGNNPRVGTKRYMAPEVLDEQIRTDCFESYKWTDIWAFGLVLWEIARRTIVNGIVEDYRPPFYDVVPNDPSFEDMKKVVCVDQQTPTIPNRLAADPVLSGLAQMMRECWYPNPSARLTALRIKKTLQKISNSPEKPKVIQ

SEQ ID NO: 2 - protein, 122 aa, synthetic construct
QVQLQQWGAGLLKPSETLSLTCAVYGGSFSSSYWNWIRQPPGKGLEWIGEVNHSGSTNYNPSLKSRVTISLDTSKNQFSLKLSSVTAADTAVYYCARSPRSGRIVGAVFDYWGQGTLVTVSS

SEQ ID NO: 3 - protein, 108 aa, synthetic construct
SYVLTQPPSVSVAPGQTARITCGGNNIGPKSVHWYQQKPGQAPVLVVYDDSDRPSGIPERFSGSNSGNTATLTISRVEAGDEADYYCQVWDSSNDHVVFGGGTKLTVL

SEQ ID NO: 4 - protein, 121 aa, synthetic construct
QLQLQESGPGLVKPSETLSLTCTVSGGSISSRSYYWGWIRQPPGKGLEWIGNIYYSGSAFYNPSLKSRVTISVDTSKNQFSLKLSSVTAADTAVYYCARWDNWDVGAFDIWGQGTMVTVSS

SEQ ID NO: 5 - protein, 110 aa, synthetic construct
QSALTQPPSASGSPGQSVTISCTGTSSDVGGYNYVSWYQQHPGKAPKLMIYEVSKRPSGVPDRFSGSKSGNTASLTVSGLQAEDEADYYCTSFAGSNNWVFGGGTKLTVL

SEQ ID NO: 6 - protein, 120 aa, synthetic construct
QVQLQESGPGLVKPSQTLSLTCTVSGGSISSSGHYWSWIRQHPGKGLEWIGYIYYSGDTYYNPSLKSRITMSVDTSKNQFSLKLSSVTAADTAVYYCARPKLDDWALDYWGQGTLVTVSS

SEQ ID NO: 7 - protein, 108 aa, synthetic construct
SYVLTQPPSVSVAPGQTARITCGGNNIGSKTVHWYQQKPGQAPVLVVYNDGDRPSGIPERFSGSNSGNTATLTISRVEAGDEADYYCQVWDSSSARVVFGGGTKLTVL

SEQ ID NO: 8 - protein, 118 aa, synthetic construct
EVQLLESGGGLVQPGGSLRLSCAASGFTFSTYAMNWVRQAPGKGLEWVSAISGRGGSTYYADSVKGRFTISRDNSKNTLYLQMNSLRAEDTAVYYCAKRGLGGPFDYWGQGTLVTVSS

SEQ ID NO: 9 - protein, 107 aa, synthetic construct
DIQMTQSPSSLSASVGDRVTITCRASQGISNYLAWFQQKPGKAPKSLIYGASSLQSGVPSKFSGSGSGTDFTLTISSLQPEDFATYYCQQYNSYPRTFGQGTKVEIK

SEQ ID NO: 10 - protein, 117 aa, synthetic construct
EVQLLESGGGLVQPGGSLRLSCAASGFAFNSFAMSWVRQAPGKGLDWVSAIGGRGGSTYYADSVKGRFTISRDNSKNTLFLQMNSLRAEDTAVYYCASQPTPPFDYWGQGTLVTVSS

SEQ ID NO: 11 - protein, 107 aa, synthetic construct
AIQMTQSPSSLSASVGDRVTITCRASQDIRNDLGWYQQRPGKAPQLLIYATSILQSGVPSRFSGSGSGTVFTLTISSLQPEDFATYYCLQDYNYPCNFGQGTKLEIK

SEQ ID NO: 12 - protein, 117 aa, synthetic construct
QVQLVQSGAEVKKPGASVKVSCKASGYTFTGYYIHWVRQAPGQGLEWMGRIVPKSGXTNYAQKFQGRVTMTRDTSISAAYMELSRLRSDDTAMYYCARDGQWLPDYWSQGTLVTVSS

SEQ ID NO: 13 - protein, 107 aa, synthetic construct
DIQMTQSPSSLSASVGDRVTITCRASQGIRNDLGWYQQKPGKAPKRLIYAASSLHSGVPSRFSGSGSGTEFTLTISSLQPEDFATYYCLQHNSYPYTFGQGTRLDIK

SEQ ID NO: 14 - DNA, 366 bp, synthetic construct
  source 1..366: mol_type = other DNA
caggtgcagctacagcagtggggcgcaggactgttgaagccttcggagaccctgtccctcacctgcgctgtctatggtgggtccttcagtagttcctactggaactggatccgccagcccccagggaagggactggagtggattggggaagtcaatcatagtggaagcaccaactacaacccgtccctcaagagtcgagtcaccatatcactagacacgtccaagaaccagttctccctgaagctgagctctgtgaccgccgcggacacggctgtgtattactgtgcgagatcgcctagatcgggccgtatagtgggagccgtctttgactactggggccagggaaccctggtcaccgtctcctca

SEQ ID NO: 15 - DNA, 324 bp, synthetic construct
  source 1..324: mol_type = other DNA
tcctatgtgctgactcagccaccctcggtgtcagtggccccaggacagacggccaggattacctgtgggggaaacaacattggacctaaaagtgtgcactggtaccagcagaagccaggccaggcccctgtgttggtcgtctatgatgatagcgaccggccctcagggatccctgagcgattctctggctccaactctgggaacacggccaccctgaccatcagcagggtcgaagccggggatgaggccgactattactgtcaggtgtgggatagtagtaatgatcatgtagtattcggcggagggaccaagctgaccgtccta

SEQ ID NO: 16 - DNA, 363 bp, synthetic construct
  source 1..363: mol_type = other DNA
cagctgcagctgcaggagtcgggcccaggactggtgaagccttcggagaccctgtccctcacttgcactgtctctggtggctccatcagcagtagaagttactactggggctggatccgccagccccccgggaaggggctagaatggattgggaatatctattatagtgggagcgccttctacaacccgtccctcaagagtcgagtcaccatatccgtagacacgtccaagaaccagttctccctgaagctgagctctgtgaccgccgcagacacggctgtgtattactgtgcgagatgggataactgggacgtaggagcttttgatatctggggccaagggacaatggtcaccgtctcttca

SEQ ID NO: 17 - DNA, 330 bp, synthetic construct
  source 1..330: mol_type = other DNA
cagtctgccctgactcagcctccctccgcgtccgggtctcctggacagtcagtcaccatctcctgcactggaaccagcagtgacgttggtggttataactatgtctcctggtaccaacagcacccaggcaaagcccccaaactcatgatttatgaggtcagtaagcggccctcaggggtccctgatcgcttctctggctccaagtctggcaacacggcctccctgaccgtctctgggctccaggctgaggatgaggctgattattactgcacctcatttgcaggcagcaacaattgggtgttcggcggagggaccaagctgaccgtccta

SEQ ID NO: 18 - DNA, 360 bp, synthetic construct
  source 1..360: mol_type = other DNA
caggtgcagctgcaggagtcgggcccaggactggtgaagccttcacagaccctgtccctcacctgcactgtctctggtggctccatcagcagtagtggtcactactggagctggatccgccagcacccagggaagggcctggagtggattgggtacatctattacagtggggacacctactacaacccgtccctcaagagtcgaattaccatgtcagtagacacgtctaagaaccagttctccctgaagctgagctctgtgactgccgcggacacggccgtatattactgtgcgagaccgaagttagatgactgggcccttgactactggggccagggaaccctggtcaccgtctcctca

SEQ ID NO: 19 - DNA, 324 bp, synthetic construct
  source 1..324: mol_type = other DNA
tcctatgtgctgactcagccaccctcggtgtcagtggccccaggacagacggccaggattacctgtgggggaaataacattggaagtaaaactgtgcactggtaccagcagaagccaggccaggcccctgtgctggtcgtctataatgatggcgaccggccctcagggatccctgagcgattctctggctccaactctgggaacacggccaccctgaccatcagcagggtcgaagccggggatgaggccgactactactgtcaggtgtgggatagtagtagtgctcgtgtggtgttcggcggagggaccaagctgaccgtccta

SEQ ID NO: 20 - DNA, 354 bp, synthetic construct
  source 1..354: mol_type = other DNA
gaggtgcagctgttggagtctgggggaggcttggtacagcctggggggtccctgagactctcctgtgcagcctctggattcacctttagcacctatgccatgaactgggtccgccaggctccggggaaggggctggagtgggtctcagctattagtggtcgtggtggtagcacatactacgcagactccgtgaagggccggttcaccatctccagagacaattccaagaacacgctgtatctgcaaatgaacagcctgagagccgaggacacggccgtatattactgtgcgaaaagggggctggggggtccatttgactactggggccagggaaccctggtcaccgtctcctca

SEQ ID NO: 21 - DNA, 321 bp, synthetic construct
  source 1..321: mol_type = other DNA
gacatccagatgacccagtctccatcctcactgtctgcatctgtaggagacagagtcaccatcacttgtcgggcgagtcagggcattagcaattatttagcctggtttcagcagaaaccagggaaagcccctaagtccctgatctatggtgcatccagtttgcaaagtggggtcccatcaaagttcagcggcagtggatctgggacagatttcactctcaccatcagcagcctgcagcctgaagattttgcaacttattactgccaacagtataatagttaccctcggacgttcggccaagggaccaaggtggaaatcaaa

SEQ ID NO: 22 - DNA, 351 bp, synthetic construct
  source 1..351: mol_type = other DNA
gaggtccagctgttggagtctgggggaggcttggtccagcctggggggtccctgagactctcctgtgcagcctctggattcgcctttaacagctttgccatgagttgggtccgccaggctccagggaaggggctggactgggtctcagctattggtggtcgtggtggtagcacatactacgcagactccgtgaagggccggttcaccatctcccgagacaattccaagaacacgctgtttctgcaaatgaacagcctgagagccgaggacacggccgtatattactgtgcgagccaacccacgcccccctttgactactggggccagggaaccctggtcaccgtctcctca

SEQ ID NO: 23 - DNA, 321 bp, synthetic construct
  source 1..321: mol_type = other DNA
gccatccagatgacccagtctccatcctccctgtctgcatctgtaggagacagagtcaccatcacttgccgggcaagtcaggacattagaaatgatttaggctggtatcagcagagaccagggaaagcccctcaactcctgatctatgctacatccattttacaaagtggggtcccatcaaggttcagcggcagtggatctggcacagttttcactctcaccatcagcagcctgcagcctgaagattttgcgacttattactgtctacaagattacaattacccgtgcaattttggccaggggaccaagctggagatcaaa

SEQ ID NO: 24 - DNA, 351 bp, synthetic construct
  source 1..351: mol_type = other DNA
caggtgcagctggtgcagtctggggctgaggtgaagaagcctggggcctcagtgaaggtctcctgcaaggcttctggatacaccttcaccggctactatatccactgggtgcgacaggcccctggacaagggcttgagtggatgggacggatcgtccctaaaagtggtgtcacaaactatgcacagaagtttcagggcagggtcaccatgaccagggacacgtccatcagcgcagcctacatggagctgagcaggctgagatctgacgacacggccatgtattactgtgcgagagacggtcagtggctgcctgactactggagccagggaaccctggtcaccgtctcctca

SEQ ID NO: 25 - DNA, 321 bp, synthetic construct
  source 1..321: mol_type = other DNA
gacatccagatgacccagtctccatcctccctgtctgcatctgtaggagacagagtcaccatcacttgccgggcaagtcagggcattagaaatgatttaggctggtatcaacagaaaccagggaaagcccctaagcgcctgatctatgctgcatccagtttgcacagtggggtcccatcaaggttcagcggcagtggatctgggacagaattcactctcacaatcagcagcctgcagcctgaagattttgcaacttattactgtctacagcataatagttacccgtacacttttggccaggggaccaggctggacatcaaa

SEQ ID NO: 26 - protein, 5 aa, synthetic construct
SSYWN

SEQ ID NO: 27 - protein, 16 aa, synthetic construct
EVNHSGSTNYNPSLKS

SEQ ID NO: 28 - protein, 14 aa, synthetic construct
SPRSGRIVGAVFDY

SEQ ID NO: 29 - protein, 11 aa, synthetic construct
GGNNIGPKSVH

SEQ ID NO: 30 - protein, 7 aa, synthetic construct
DDSDRPS

SEQ ID NO: 31 - protein, 11 aa, synthetic construct
QVWDSSNDHVV

SEQ ID NO: 32 - protein, 7 aa, synthetic construct
SRSYYWG

SEQ ID NO: 33 - protein, 16 aa, synthetic construct
NIYYSGSAFYNPSLKS

SEQ ID NO: 34 - protein, 11 aa, synthetic construct
WDNWDVGAFDI

SEQ ID NO: 35 - protein, 14 aa, synthetic construct
TGTSSDVGGYNYVS

SEQ ID NO: 36 - protein, 7 aa, synthetic construct
EVSKRPS

SEQ ID NO: 37 - protein, 10 aa, synthetic construct
TSFAGSNNWV

(19) United States
(12) Patent Application Publication
(10) Pub. No.: US 2025/0154264 A1
(43) Pub. Date: May 15, 2025

(54) ANTI-ALK1 ANTIBODIES AND METHODS OF USING THE SAME

(71) Applicants: Genovac Antibody Discovery LLC, Fargo, ND (US); Yale University, New Haven, CT (US)

(72) Inventors: Hubertus Schleer, Fargo, ND (US); William Sessa, Madison, CT (US)

(73) Assignees: Genovac Antibody Discovery LLC, Fargo, ND (US); Yale University, New Haven, CT (US)

(21) Appl. No.: 18/832,414

(22) PCT Filed: Jan. 23, 2023
(86) PCT No.: PCT/US2023/011339

§ 371 Date: Jul. 23, 2024

Related U.S. Application Data
(60) Provisional application No. 63/335,444, filed on Apr. 27, 2022.
(60) Provisional application No. 63/302,408, filed on Jan. 24, 2022.

(51) Int. Cl. C07K 16/28; A61K 39/00; A61P 9/10

(52) U.S. Cl. CPC C07K 16/2863; A61P 9/10; A61K 2039/505; C07K 2317/33; C07K 2317/76; C07K 2317/92

(57) ABSTRACT

The present disclosure provides antibodies that bind to ALK1 and methods/uses of the disclosed antibodies for treating various conditions (e.g., atherosclerosis). The present disclosure provides anti-ALK1 antibodies, comprising the complementarity determining regions (CDRs) of a heavy chain variable sequence and a light chain variable sequence selected from: the heavy chain variable sequence of SEQ ID NO: 2 and the light chain variable sequence of SEQ ID NO: 3. In some embodiments, the disclosed antibodies bind to human ALK1, murine ALK1, cynomolgus ALK1, or a combination thereof. The present disclosure also provides pharmaceutical compositions comprising an anti-ALK1 antibody accordingly to any one of the foregoing aspects or embodiments and a pharmaceutically acceptable carrier.

### CROSS-REFERENCE TO RELATED APPLICATION

This application claims priority to U.S. Provisional Application No. 63/302,408, filed Jan. 24, 2022, and U.S. Provisional Application No. 63/335,444, filed Apr. 27, 2022, the disclosures of which are hereby incorporated by reference in their entireties.

### FIELD OF THE INVENTION

Antibodies that bind to Activin A receptor like type 1 (ALK1) are provided. Treatments and uses comprising administering such antibodies are also provided.

### BACKGROUND

The following discussion is provided to aid the reader in understanding the disclosure and is not admitted to describe or constitute prior art thereto.

Activin receptor-like kinase-1 or ALK1 is a type I cell surface receptor for the transforming growth factor-β (TGF-β) family of proteins. The role of ALK1 in endothelial cells biology and in angiogenesis has been thoroughly studied. However, it has been recently suggested a possible role of ALK1 in cardiovascular homeostasis. Indeed, due to the ability of ALK1 to regulate cell proliferation and migration, and to modulate extracellular matrix (ECM) protein expression in several cell types, ALK1 has been now demonstrated to play an important role in cardiovascular remodeling.

ALK1 is not only expressed in endothelial cells but also in smooth muscle cells, myofibroblast, hepatic stellate cells, chondrocytes, monocytes, myoblasts, macrophages or fibroblasts, but its role in these cells have not been deeply analyzed. Due to the function of ALK1 in these cells, this receptor plays a role in several cardiovascular diseases, such as atherosclerosis. Cardiovascular diseases, in general, and atherosclerosis, specifically, are the leading causes of death worldwide.

Accordingly, there remains a need for ALK1-targeting therapies, such as antibodies that bind ALK1, for treatment of atherosclerosis and other diseases and disorders.

### SUMMARY

The present disclosure provides antibodies that bind to ALK1 and methods/uses of the disclosed antibodies for treating various conditions (e.g., atherosclerosis).

In one aspect, the present disclosure provides anti-ALK1 antibodies, comprising the complementarity determining regions (CDRs) of a heavy chain variable sequence and a light chain variable sequence selected from: the heavy chain variable sequence of SEQ ID NO: 2 and the light chain variable sequence of SEQ ID NO: 3; the heavy chain variable sequence of SEQ ID NO: 4 and the light chain variable sequence of SEQ ID NO: 5; the heavy chain variable sequence of SEQ ID NO: 6 and the light chain variable sequence of SEQ ID NO: 7; the heavy chain variable sequence of SEQ ID NO: 10 and the light chain variable sequence of SEQ ID NO: 11; and the heavy chain variable sequence of SEQ ID NO: 12 and the light chain variable sequence of SEQ ID NO: 13.

The antibodies may further comprise a variable heavy chain sequence and a variable light chain sequence selected from: the heavy chain variable sequence of SEQ ID NO: 2 and the light chain variable sequence of SEQ ID NO: 3; the heavy chain variable sequence of SEQ ID NO: 4 and the light chain variable sequence of SEQ ID NO: 5; the heavy chain variable sequence of SEQ ID NO: 6 and the light chain variable sequence of SEQ ID NO: 7; the heavy chain variable sequence of SEQ ID NO: 10 and the light chain variable sequence of SEQ ID NO: 11; and the heavy chain variable sequence of SEQ ID NO: 12 and the light chain variable sequence of SEQ ID NO: 13.

In some embodiments, the heavy chain variable sequence comprises at least 90%, 91%, 92%, 93%, 94%, 95%, 96%, 97%, 98%, 99%, or 100% sequence identity to SEQ ID NO: 2, 4, 6, 10, or 12, and the light chain variable sequence comprises at least 90%, 91%, 92%, 93%, 94%, 95%, 96%, 97%, 98%, 99%, or 100% sequence identity to SEQ ID NO: 3, 5, 7, 11, or 13.

In some embodiments, the heavy chain variable sequence comprises 1-10 amino acid substitutions, insertions or deletions in SEQ ID NO: 2, 4, 6, 10, or 12, and the light chain variable sequence comprises 1-10 amino acid substitutions, insertions or deletions in SEQ ID NO: 3, 5, 7, 11, or 13.

In another aspect, the present disclosure provideds anti-ALK1 antibodies comprising a heavy chain comprising a CDRH1 comprising SSYWN (SEQ ID NO: 26), a CDRH2 comprising EVNHSGSTNYNPSLKS (SEQ ID NO: 27), and a CDRH3 comprising SPRSGRIVGAVFDY (SEQ ID NO: 28); and a light chain comprising a CDRL1 comprising GGNNIGPKSVH (SEQ ID NO: 29), a CDRL2 comprising DDSDRPS (SEQ ID NO: 30), and a CDRL2 comprising QVWDSSNDHVV (SEQ ID NO: 31).

In another aspect, the present disclosure provideds anti-ALK1 antibodies comprising a heavy chain comprising a CDRH1 comprising SRSYYWG (SEQ ID NO: 32), a CDRH2 comprising NIYYSGSAFYNPSLKS (SEQ ID NO: 33), and a CDRH3 comprising WDNWDVGAFDI (SEQ ID NO: 34); and a light chain comprising a CDRL1 comprising TGTSSDVGGYNYVS (SEQ ID NO: 35), a CDRL2 comprising EVSKRPS (SEQ ID NO: 36), and a CDRL2 comprising TSFAGSNNWV (SEQ ID NO: 37).

In some embodiments, the disclosed antibodies bind to human ALK1, murine ALK1, cynomolgus ALK1, or a combination thereof. In some embodiments, the disclosed antibodies do not bind to ALK5. In some embodiments, the disclosed antibodies do not block BMP9 signaling.

The present disclosure also provides pharmaceutical compositions comprising an anti-ALK1 antibody accordingly to any one of the foregoing aspects or embodiments and a pharmaceutically acceptable carrier. In some embodiments, the pharmaceutical composition can be formulated for injection or infusion.

In another aspect, the present disclosure provides methods of treating or preventing a disease or disorder associated with ALK1, comprising administering to a subject with a disease or disorder associated with ALK1 an antibody or pharmaceutical composition according to any one of the foregoing aspects or embodiments.

In some embodiments, the disease is atherosclerosis. In some embodiments, the risk of developing atherosclerosis is reduced and the subject that has not yet developed atherosclerotic plaques. In some embodiments, further atherosclerotic plaque formation is reduced or inhibited. In some embodiments, existing atherosclerotic plaques are destroyed. In some embodiments, the size or number of existing atherosclerotic plaques is reduced.

In some embodiments, the anti-ALK1 antibody or pharmaceutical composition is administered intravenously, subcutaneously, intramuscularly, or intraperitoneally. In some embodiments, the subject is human.

In another aspect, the present disclosure provides methods of blocking an interaction between ALK1 and a low density lipoprotein (LDL), comprising administering to a subject an antibody or pharmaceutical composition according to any one of the foregoing aspects or embodiments. In some embodiments, the subject has atherosclerosis or is at risk of developing atherosclerosis. In some embodiments, BMP9 signaling is not inhibited. In some embodiments, the subject is human.

In another aspect, the present disclosure provides uses of any of the antibodies disclosed herein for the manufacture of a medicament for treating a disease associated with ALK1, such as atherosclerosis.

In another aspect, the present disclosure provides uses of any of the antibodies disclosed herein for the manufacture of a medicament for blocking an interaction between ALK1 and a low density lipoprotein (LDL).

In another aspect, the present disclosure provides anti-ALK1 antibodies for use in treating a disease associated with ALK1, such as atherosclerosis.

In another aspect, the present disclosure provides anti-ALK1 antibodies for use in blocking an interaction between ALK1 and a low density lipoprotein (LDL).

The foregoing general description and following detailed description are exemplary and explanatory and are intended to provide further explanation of the disclosure as claimed. Other objects, advantages, aspects and iterations of the disclosure are provided in more detail below.

### BRIEF DESCRIPTION OF THE DRAWINGS

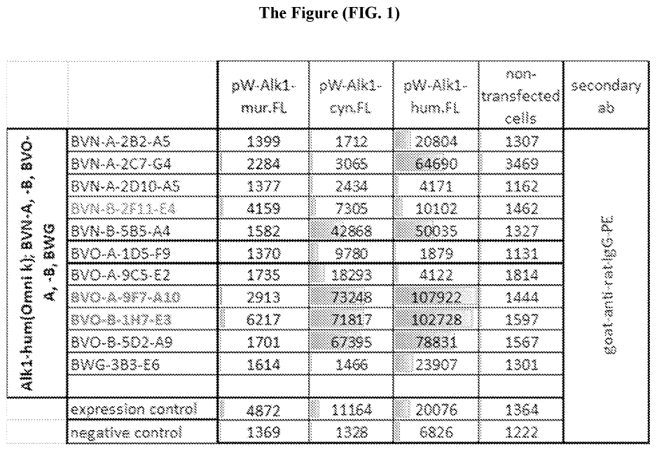

The FIG. 1 shows results of ALK1 binding assays to confirm binding of disclosed antibodies to various mammalian orthologs of ALK1.

### DETAILED DESCRIPTION

Antibodies that bind Activin A receptor like type 1 (ALK1) are provided. Antibody heavy chains and light chains that are capable of forming antibodies that bind ALK1 are also provided. In addition, antibodies, heavy chains, and light chains comprising one or more particular complementarity determining regions (CDRs) are provided. Polynucleotides encoding antibodies that bind to ALK1 are provided. Polynucleotides encoding antibody heavy chains or lights chains are also provided. Methods of producing and/or purifying antibodies to ALK1 are provided. Treatments using the antibodies are provided. Such methods include, but are not limited to, methods of treating atherosclerosis.

### I. Definitions

It is to be understood that the terminology used herein is for the purpose of describing particular embodiments only, and is not intended to be limiting.

Technical and scientific terms used herein have the meanings commonly understood by one of ordinary skill in the art, unless otherwise defined. Unless otherwise specified, materials and/or methodologies known to those of ordinary skill in the art can be utilized in carrying out the methods described herein, based on the guidance provided herein.

As used herein, the singular terms “a,” “an,” and “the” include plural referents unless the context clearly dictates otherwise. Reference to an object in the singular is not intended to mean “one and only one” unless explicitly so stated, but rather “one or more.”

As used herein, “about” when used with a numerical value means the numerical value stated as well as plus or minus 10% of the numerical value. For example, “about 10” should be understood as both “10” and “9-11.”

Also as used herein, “and/or” refers to and encompasses any and all possible combinations of one or more of the associated listed items, as well as the lack of combinations when interpreted in the alternative (“or”).

As used herein, a phrase in the form “A/B” or in the form “A and/or B” means (A), (B), or (A and B); a phrase in the form “at least one of A, B, and C” means (A), (B), (C), (A and B), (A and C), (B and C), or (A, B, and C).

As used herein, the phrase “therapeutically effective amount” with reference to an anti-ALK1 antibody means that dose of the antibody that provides the specific pharmacological effect for which the drug is administered in a subject in need of such treatment. For example, a therapeutically effective amount may be effective to reduce, ameliorate, or eliminate plaque buildup or signs and symptoms of atherosclerosis. It is emphasized that a therapeutically effective amount of an anti-ALK1 antibody will not always be effective in treating the disease or condition (e.g., atherosclerosis) in every individual subject, even though such dose is deemed to be a therapeutically effective amount by those of skill in the art. Those skilled in the art can adjust what is deemed to be a therapeutically effective amount in accordance with standard practices as needed to treat a specific subject. A therapeutically effective amount may vary based on, for example, the age and weight of the subject, and/or the subject's overall health, and/or the severity of the subject's disease or condition (e.g., atherosclerosis).

The terms “treat,” “treatment” or “treating” as used herein with reference to a disease or disorder associated with ALK1 (e.g., atherosclerosis) refer to reducing, ameliorating, or eliminating pathophysiological features of the disease or condition (e.g., plaque formation in atherosclerosis), reducing, ameliorating, or eliminating signs or symptoms of the disease or disorder, and/or otherwise improving quality of life in a subject. For example, in the case of atherosclerosis, treatment may entail improving blood flow, decreasing blood pressure, inhibiting deposition of cholesterol plaques in/on endothelial lining of vessels, breaking up existing plaques, or a combination of any or all of the foregoing.

The terms “prevent,” “preventing” or “prevention” as used herein with reference to a disease or disorder associated with ALK1 (e.g., atherosclerosis) refer to precluding or reducing the risk of developing the disease or disorder. For example, preventing atherosclerosis may entail preventing the formation of plaques in an individual predisposed to plaque formation or reducing the risk of plaque formation in such an individual.

The terms “individual,” “subject,” and “patient” are used interchangeably herein, and refer to any individual mammalian subject, e.g., bovine, canine, feline, equine, or human. In specific embodiments, the subject, individual, or patient is a human.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to an ALK1 epitope is an antibody that binds this epitope with greater affinity, avidity, more readily, and/or with greater duration than it binds to other ALK1 epitopes or non-ALK1 epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

The term “antibody” herein is used in the broadest sense and encompasses various antibody structures, including but not limited to monoclonal antibodies, polyclonal antibodies, multispecific antibodies (for example, bispecific (such as Bi-specific T-cell engagers) and trispecific antibodies), and antibody fragments so long as they exhibit the desired antigen-binding activity.

The term “CDR” denotes a complementarity determining region as defined by at least one manner of identification to one of skill in the art.

The term “heavy chain variable region” as used herein refers to a region comprising at least three heavy chain CDRs. In some embodiments, the heavy chain variable region includes the three CDRs and at least FR2 and FR3. In some embodiments, the heavy chain variable region includes at least heavy chain HCDR1, framework (FR) 2, HCDR2, FR3, and HCDR3. In some embodiments, a heavy chain variable region also comprises at least a portion of an FRI and/or at least a portion of an FR4.

The term “heavy chain constant region” as used herein refers to a region comprising at least three heavy chain constant domains, C_{H}1, C_{H}2, and C_{H}3. Of course, non-function-altering deletions and alterations within the domains are encompassed within the scope of the term “heavy chain constant region,” unless designated otherwise. Non-limiting exemplary heavy chain constant regions include γ, δ, and α. Non-limiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an a constant region is an IgA antibody. Further, an antibody comprising a μconstant region is an IgM antibody, and an antibody comprising an ε constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” as used herein refers to a region comprising at least three light chain CDRs. In some embodiments, the light chain variable region includes the three CDRs and at least FR2 and FR3. In some embodiments, the light chain variable region includes at least light chain LCDR1, framework (FR) 2, LCDR2, FR3, and LCDR3. For example, a light chain variable region may comprise light chain CDR1, framework (FR) 2, CDR2, FR3, and CDR3. In some embodiments, a light chain variable region also comprises at least a portion of an FRI and/or at least a portion of an FR4.

The term “light chain constant region” as used herein refers to a region comprising a light chain constant domain, C_{L}. Non-limiting exemplary light chain constant regions include λ and κ. Of course, non-function-altering deletions and alterations within the domains are encompassed within the scope of the term “light chain constant region,” unless designated otherwise.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

“Affinity” refers to the strength of the sum total of noncovalent interactions between a single binding site of a molecule (for example, an antibody) and its binding partner (for example, an antigen). The affinity of a molecule X for its partner Y can generally be represented by the dissociation constant (K_{D}). Affinity can be measured by common methods known in the art (such as, for example, ELISA K_{D}, KinExA, bio-layer interferometry (BLI), and/or surface plasmon resonance devices (such as a BIAcore® device), including those described herein).

The term “K_{D}”, as used herein, refers to the equilibrium dissociation constant of an antibody-antigen interaction.

A “chimeric antibody” as used herein refers to an antibody in which a portion of the heavy and/or light chain is derived from a particular source or species, while at least a part of the remainder of the heavy and/or light chain is derived from a different source or species. In some embodiments, a chimeric antibody refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, all of the variable regions of a chimeric antibody are from a first species and all of the constant regions of the chimeric antibody are from a second species. The chimeric construct can also be a functional fragment, as noted above.

A “humanized antibody” as used herein refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is an antibody fragment, such as Fab, an scFv, a (Fab′)₂, etc. The term humanized also denotes forms of non-human (for example, murine) antibodies that are chimeric immunoglobulins, immunoglobulin chains, or fragments thereof (such as Fv, Fab, Fab′, F(ab′)₂ or other antigen-binding subsequences of antibodies) that contain minimal sequence of non-human immunoglobulin. Humanized antibodies can include human immunoglobulins (recipient antibody) in which residues from a complementary determining region (CDR) of the recipient are substituted by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody can comprise residues that are found neither in the recipient antibody nor in the imported CDR or framework sequences, but are included to further refine and optimize antibody performance. In general, the humanized antibody can comprise substantially all of at least one, and typically two, variable domains, in which all or substantially all of the CDR regions correspond to those of a non-human immunoglobulin and all or substantially all of the FR regions are those of a human immunoglobulin consensus sequence. In some embodiments, the humanized antibody can also comprise at least a portion of an immunoglobulin constant region or domain (Fc), typically that of a human immunoglobulin. Other forms of humanized antibodies have one or more CDRs (CDR L1, CDR L2, CDR L3, CDR HI, CDR H2, and/or CDR H3) which are altered with respect to the original antibody, which are also termed one or more CDRs “derived from” one or more CDRs from the original antibody. As will be appreciated, a humanized sequence can be identified by its primary sequence and does not necessarily denote the process by which the antibody was created.

An “CDR-grafted antibody” as used herein refers to a humanized antibody in which one or more complementarity determining regions (CDRs) of a first (non-human) species have been grafted onto the framework regions (FRs) of a second (human) species.

A “human antibody” as used herein encompasses antibodies produced in humans, antibodies produced in non-human animals that comprise human immunoglobulin genes, such as XenoMouse® mice, and antibodies selected using in vitro methods, such as phage display (Vaughan et al., 1996, Nature Biotechnology, 14:309-314; Sheets et al., 1998, Proc. Natl. Acad. Sci. (USA) 95:6157-6162; Hoogenboom and Winter, 1991, J. Mol. Biol., 227:381; Marks et al., 1991, J. Mol. Biol., 222:581), wherein the antibody repertoire is based on a human immunoglobulin sequence. The term “human antibody” denotes the genus of sequences that are human sequences. Thus, the term is not designating the process by which the antibody was created, but the genus of sequences that are relevant.

An amino acid substitution may include but are not limited to the replacement of one amino acid in a polypeptide with another amino acid. Exemplary conservative substitutions are shown in Table 1. Amino acid substitutions may be introduced into an antibody of interest and the products screened for a desired activity, for example, retained/improved antigen binding, decreased immunogenicity, or improved ADCC or CDC.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, B-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E and DG44 cells, respectively.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

A “sterile” formulation is aseptic or essentially free from living microorganisms and their spores.

### II. ALK1 and ALK1-Related Diseases

Activin A receptor like type 1 (ALK1; Entrez: 94, Ensembl: ENSG00000139567, UniProt: P37023) is a transmembrane serine/threonine receptor kinase in the transforming growth factor-beta receptor family that is expressed on endothelial cells. Ligands within the transforming growth factor-β (TGFβ) superfamily signal via transmembrane receptor complexes and control myriad processes important in development and disease, including embryonic axis patterning, heart and vascular development, epithelial-mesenchymal transition, and fibrosis. Among the signaling receptors, activin A receptor like type 1 (ACVRL1, which encodes ALK1) possesses a high degree of cell-type specificity, with predominant expression in arterial endothelial cells (ECs). The amino acid sequence of human ALK1 is:

(SEQ ID NO: 1)
MTLGSPRKGLLMLLMALVTQGDPVKPSRGPLVTCTCESPHCKGPTC
RGAWCTVVLVREEGRHPQEHRGCGNLHRELCRGRPTEFVNHYCCD
SHLCNHNVSLVLEATQPPSEQPGTDGQLALILGPVLALLALVALG
VLGLWHVRRRQEKQRGLHSELGESSLILKASEQGDSMLGDLLDSD
CTTGSGSGLPFLVQRTVARQVALVECVGKGRYGEVWRGLWHGESV
AVKIFSSRDEQSWFRETEIYNTVLLRHDNILGFIASDMTSRNSST
QLWLITHYHEHGSLYDFLQRQTLEPHLALRLAVSAACGLAHLHVE
IFGTQGKPAIAHRDFKSRNVLVKSNLQCCIADLGLAVMHSQGSDY
LDIGNNPRVGTKRYMAPEVLDEQIRTDCFESYKWTDIWAFGLVLW
EIARRTIVNGIVEDYRPPFYDVVPNDPSFEDMKKVVCVDQQTPTI
PNRLAADPVLSGLAQMMRECWYPNPSARLTALRIKKTLQKISNSP
EKPKVIQ

Defects in ALK1 signaling can cause the autosomal dominant vascular disorder, hereditary hemorrhagic telangiectasia (HHT), which is characterized by development of direct connections between arteries and veins, or arteriovenous malformations (AVMs). Further, it is now understood that ALK1 signaling can play a role in other vascular-associated diseases such as pulmonary arterial hypertension, cancer, and atherosclerosis. Additionally, ALK1 has been implicated in various aspects of sprouting angiogenesis, including tip/stalk cell selection, migration, and proliferation, and recent work suggests a role for ALK1 in transducing a flow-based signal that governs directed endothelial cell migration within patent, perfused vessels.

Studies in mice have demonstrated strong Alk1 expression at arterial curvatures and bifurcations, which are sites of disturbed flow that are prone to atherosclerosis. Increased ALK1 expression in the endothelium, neointima, and media of human coronary artery atherosclerotic lesions has also been demonstrated. It is now understood that ALK1 is directly implicated in atherosclerosis via a mechanism seemingly unrelated to its role in HHT.

Briefly, the apically localized extracellular domain of ALK1 can bind to circulating ApoB100-containing low density lipoproteins (LDL) with relatively low affinity and mediate transcytosis of LDL to the subendothelial space, initiating an atherosclerotic lesion. This activity does not require BMP9/BMP10, endoglin, BMPRII, or ALK1 kinase activity, and BMP9/BMP10 cannot compete out LDL binding, suggesting that these ligands bind to different ALK1 residues. As such, targeting LDL/ALK1 interaction may be a viable approach to development of new drugs to prevent atherosclerosis.

As provided in more detail below, the disclosed antibodies may be used for treating diseases and disorders associated with ALK1, such as atherosclerosis and any of the other diseases or disorders included herein (e.g., HHT, AVMs, pulmonary arterial hypertension, cancer, etc.).

### III. Anti-ALK1 Antibodies

The present disclosure provides antibodies that bind to ALK1 (i.e., “anti-ALK1 antibodies”). The disclosed antibodies can bind selectively to ALK1 and be used to treat diseases or disorders associated with ALK1, including but not limited to atherosclerosis, HHT, AVMs, pulmonary arterial hypertension, and cancer.

ALK1 can play an important role in the buildup of LDL cholesterol in blood vessels. The disclosed antibodies (e.g., BVO-A-9F7-A10 and BVO-B-1H7-E3) are able to block LDL accumulation by disrupting the “LDL-ALK1 pathway” in the walls of blood vessels that facilitates the transport of LDL from blood into tissue (aka LDL transcytosis). This in turn can help prevent or slow the clogging of arteries that leads to heart disease. The conformation selective ALK1 binding monoclonal antibodies disclosed herein (e.g., BVO-A-9F7-A10 and BVO-B-1H7-E3) efficiently block LDL transcytosis into the endothelium, but not BMP9 signaling, resulting in a dramatic reduction in plaque formation. The disclosed anti-ALK1 antibodies may bind to human ALK1, murine ALK1, cynomolgus ALK1, or a combination thereof.

Anti-ALK1 antibodies described herein can be obtained by any means, including in vitro sources (e.g., a hybridoma or a cell line producing an antibody recombinantly) and in vivo sources (e.g., rodents, rabbits, humans, etc.). The disclosed antibodies may be human, humanized (partially or fully), or chimeric. Human, partially humanized, fully humanized, and chimeric antibodies can be made by methods known in the art, such as using a transgenic animal (e.g., a mouse) wherein one or more endogenous immunoglobulin genes are replaced with one or more human immunoglobulin genes. Examples of transgenic mice wherein endogenous antibody genes are effectively replaced with human antibody genes include, but are not limited to, the HUMAB-MOUSE™, the Kirin TC MOUSE™, and the KM-MOUSE™ (see, e.g., Lonberg, Nat. Biotechnol., 23 (9): 1117-25 (2005), and Lonberg, Handb. Exp. Pharmacol., 181:69-97 (2008)).

Anti-Alk1 antibodies disclosed herein generally will be monoclonal, recombinant, or both. Monoclonal antibodies (mAbs) may obtained by methods known in the art, for example, by fusing antibody-producing cells with immortalized cells to obtain a hybridoma, and/or by generating mAbs from mRNA extracted from bone marrow, B cells, and/or spleen cells of immunized animals using combinatorial antibody library technology and/or by isolating monoclonal antibodies from serum from subjects immunized with an ALK1 antigen. Recombinant antibodies may be obtained by methods known in the art, for example, using phage display technologies, yeast surface display technologies (Chao et al., Nat. Protoc., 1 (2): 755-68 (2006)), mammalian cell surface display technologies (Beerli et al., PNAS, 105 (38): 14336-41 (2008), and/or expressing or co-expressing antibody polypeptides. Other techniques for making antibodies are known in the art, and can be used to obtain antibodies used in the methods described herein.

Typically, an antibody consists of four polypeptides: two identical copies of a heavy (H) chain polypeptide and two copies of a light (L) chain polypeptide. Typically, each heavy chain contains one N-terminal variable (V_{H}) region and three C-terminal constant (C_{H}1, C_{H}2 and C_{H}3) regions, and each light chain contains one N-terminal variable (V_{L}) region and one C-terminal constant (C_{L}) region. The variable regions of each pair of light and heavy chains form the antigen binding site of an antibody.

The term “antibody fragment,” as used herein, refer to one or more portions of an ALK1-binding antibody that exhibits the ability to bind ALK1. Examples of binding fragments include (i) Fab fragments (monovalent fragments consisting of the V_{L}, V_{H}, C_{L} and CHI domains); (ii) F(ab′)₂ fragments (bivalent fragment comprising two Fab fragments linked by a disulfide bridge at the hinge region); (iii) Fd fragments (comprising the V_{H} and CHI domains); (iv) Fv fragments (comprising the V_{L} and V_{H} domains of a single arm of an antibody), (v) dAb fragments (comprising a V_{H} domain); and (vi) isolated complementarity determining regions (CDR), e.g., V_{H} CDR3. Other examples include single chain Fv (scFv) constructs. See e.g., Bird et al., Science, 242:423-26 (1988); Huston et al., Proc. Natl. Acad. Sci. USA, 85:5879-83 (1988). Other examples include ALK1-binding domain immunoglobulin fusion proteins comprising (i) a ALK1-binding domain polypeptide (such as a heavy chain variable region, a light chain variable region, or a heavy chain variable region fused to a light chain variable region via a linker peptide) fused to an immunoglobulin hinge region polypeptide, (ii) an immunoglobulin heavy chain C_{H2} constant region fused to the hinge region, and (iii) an immunoglobulin heavy chain C_{H3} constant region fused to the C_{H2} constant region, where the hinge region may be modified by replacing one or more cysteine residues with, for example, serine residues, to prevent dimerization.

The disclosed antibodies may belong to a class of antibody selected from IgG, IgM, IgA, IgE, and IgD. More specifically, the disclosed antibodies may be an IgG1, IgG2, IgG3, or IgG4. In some embodiments, the disclosed antibodies may comprise all or part of the constant regions, framework regions, or a combination thereof of an IgG, IgM, IgA, IgE, or IgD antibody. For instance, a disclosed antibody may comprise an IgG1 immunoglobulin structure that can be modified to replace (or “switch”) the IgG1 structure with the corresponding structure of another IgG-class immunoglobulin or an IgM, IgA, IgE, or IgD immunoglobulin. This type of modification or switching may be performed in order to augment the neutralization functions of the peptide, such as antibody dependent cell cytotoxicity (ADCC) and complement fixation (CDC). In some embodiments, the anti-ALK1 antibody may be mammalian, human, humanized, or chimeric.

In some embodiments, the disclosed anti-ALK1 antibodies may comprise one or more mutations that make the antibody more suitable in a therapeutic context. Such mutations, alterations, or modifications may comprise, for example, changes to the Fc region to increase the ability of the peptide to mediate cellular cytotoxicity functions like antibody dependent cell cytotoxicity (ADCC), antibody dependent cell mediated phagocytosis (ADCP), and/or complement fixation (CDC). A wide number of mutations to the Fc domain that enhance binding to Fc receptors have been reported, for example, S239D/A330L/1332E, F243L, and G236A. Additionally or alternatively, mutations to the Fc region that increase the circulating half-life may be incorporated into the structure. For example, mutations to engineer the pH-dependent interaction of the Fc domain with FcRn to increase affinity at pH 6.0 while retaining minimal binding at pH 7.4, can increase half-life and improve efficacy under physiological conditions. In some embodiments, the disclosed antibodies may be conjugated to polyethylene glycol (PEG) and/or albumin, which may increase the half-life and decrease the potential immunogenicity of the antibody.

Variable heavy and variable light chain amino acid sequences of exemplary anti-ALK1 antibodies are disclosed in Table 2 below. Nucleic acid sequences encoding the variable heavy and light chain sequences of Table 2 are shown in Table 3.

TABLE 2

| | | SEQ |
|---|---|---|
| Antibody & Chain | AminQ Acid Sequence | ID NO: |
| BVO-A-9F7-A10 VH | QVQLQQWGAGLLKPSETLSLTCAVYGGSFSSSYWNW | 2 |
| | IRQPPGKGLEWIGEVNHSGSTNYNPSLKSRVTISLD | |
| | TSKNQFSLKLSSVTAADTAVYYCARSPRSGRIVGAV | |
| | FDYWGQGTLVTVSS | |
| BVO-A-9F7-A10 VL | SYVLTQPPSVSVAPGQTARITCGGNNIGPKSVHWYQ | 3 |
| | QKPGQAPVLVVYDDSDRPSGIPERFSGSNSGNTATL | |
| | TISRVEAGDEADYYCQVWDSSNDHVVFGGGTKLTVL | |
| BVO-B-1H7-E3 VH | QLQLQESGPGLVKPSETLSLTCTVSGGSISSRSYYWG | 4 |
| | WIRQPPGKGLEWIGNIYYSGSAFYNPSLKSRVTISVD | |
| | TSKNQFSLKLSSVTAADTAVYYCARWDNWDVGAFDIW | |
| | GQGTMVTVSS | |
| BVO-B-1H7-E3 VL | QSALTQPPSASGSPGQSVTISCTGTSSDVGGYNYVS | 5 |
| | WYQQHPGKAPKLMIYEVSKRPSGVPDRFSGSKSGNT | |
| | ASLTVSGLQAEDEADYYCTSFAGSNNWVFGGGTKLT | |
| | VL | |
| BVO-B-5D2-A9 VH | QVQLQESGPGLVKPSQTLSLTCTVSGGSISSSGHYW | 6 |
| | SWIRQHPGKGLEWIGYIYYSGDTYYNPSLKSRITMS | |
| | VDTSKNQFSLKLSSVTAADTAVYYCARPKLDDWALD | |
| | YWGQGTLVTVSS | |
| BVO-B-5D2-A9 VL | SYVLTQPPSVSVAPGQTARITCGGNNIGSKTVHWYQ | 7 |
| | QKPGQAPVLVVYNDGDRPSGIPERFSGSNSGNTATL | |
| | TISRVEAGDEADYYCQVWDSSSARVVFGGGTKLTVL | |
| BVN-B-2F11-E4 VH | EVQLLESGGGLVQPGGSLRLSCAASGFTFSTYAMNW | 8 |
| (Control) | VRQAPGKGLEWVSAISGRGGSTYYADSVKGRFTISR | |
| | DNSKNTLYLQMNSLRAEDTAVYYCAKRGLGGPFDY | |
| | WGQGTLVTVSS | |
| BVN-B-2F11-E4-VL | DIQMTQSPSSLSASVGDRVTITCRASQGISNYLAWFQ | 9 |
| (Control) | QKPGKAPKSLIYGASSLQSGVPSKFSGSGSGTDFTLT | |
| | ISSLQPEDFATYYCQQYNSYPRTFGQGTKVEIK | |
| BVN-A-2C7-G4 VH | EVQLLESGGGLVQPGGSLRLSCAASGFAFNSFAMS | 10 |
| | WVRQAPGKGLDWVSAIGGRGGSTYYADSVKGRFTI | |
| | SRDNSKNTLFLQMNSLRAEDTAVYYCASQPTPPFD | |
| | YWGQGTLVTVSS | |
| BVN-A-2C7-G4 VL | AIQMTQSPSSLSASVGDRVTITCRASQDIRNDLGWY | 11 |
| | QQRPGKAPQLLIYATSILQSGVPSRFSGSGSGTVFT | |
| | LTISSLQPEDFATYYCLQDYNYPCNFGQGTKLEIK | |
| BVN-B-5B5-A4 VH | QVQLVQSGAEVKKPGASVKVSCKASGYTFTGYYIH | 12 |
| | WVRQAPGQGLEWMGRIVPKSGXTNYAQKFQGRVTM | |
| | TRDTSISAAYMELSRLRSDDTAMYYCARDGQWLPD | |
| | YWSQGTLVTVSS | |
| BVN-B-5B5-A4 VL | DIQMTQSPSSLSASVGDRVTITCRASQGIRNDLGWY | 13 |
| | QQKPGKAPKRLIYAASSLHSGVPSRFSGSGSGTEFT | |
| | LTISSLQPEDFATYYCLQHNSYPYTFGQGTRLDIK | |

Complementarity determining regions (CDRs) are shown in bold, underlined text.
CDR annotation was made according to Kabat numbering.

TABLE 3

| | | SEQ |
|---|---|---|
| Antibody & Chain | Nucleotide Sequences | ID NO: |
| BVO-A-9F7-A10_VH | CAGGTGCAGCTACAGCAGTGGGGCGCAGGACTGTT | 14 |
| | GAAGCCTTCGGAGACCCTGTCCCTCACCTGCGCTG | |
| | TCTATGGTGGGTCCTTCAGTAGTTCCTACTGGAAC | |
| | TGGATCCGCCAGCCCCCAGGGAAGGGACTGGAGTG | |
| | GATTGGGGAAGTCAATCATAGTGGAAGCACCAACT | |
| | ACAACCCGTCCCTCAAGAGTCGAGTCACCATATCA | |
| | CTAGACACGTCCAAGAACCAGTTCTCCCTGAAGCT | |
| | GAGCTCTGTGACCGCCGCGGACACGGCTGTGTATT | |
| | ACTGTGCGAGATCGCCTAGATCGGGCCGTATAGTG | |
| | GGAGCCGTCTTTGACTACTGGGGCCAGGGAACCCT | |
| | GGTCACCGTCTCCTCA | |
| BVO-A-9F7-A10_VL | TCCTATGTGCTGACTCAGCCACCCTCGGTGTCAGTG | 15 |
| | GCCCCAGGACAGACGGCCAGGATTACCTGTGGGGG | |
| | AAACAACATTGGACCTAAAAGTGTGCACTGGTACC | |
| | AGCAGAAGCCAGGCCAGGCCCCTGTGTTGGTCGTC | |
| | TATGATGATAGCGACCGGCCCTCAGGGATCCCTGA | |
| | GCGATTCTCTGGCTCCAACTCTGGGAACACGGCCA | |
| | CCCTGACCATCAGCAGGGTCGAAGCCGGGGATGAG | |
| | GCCGACTATTACTGTCAGGTGTGGGATAGTAGTAA | |
| | TGATCATGTAGTATTCGGCGGAGGGACCAAGCTGA | |
| | CCGTCCTA | |
| BVO-B-1H7-E3_VH | CAGCTGCAGCTGCAGGAGTCGGGCCCAGGACTGGT | 16 |
| | GAAGCCTTCGGAGACCCTGTCCCTCACTTGCACTGT | |
| | CTCTGGTGGCTCCATCAGCAGTAGAAGTTACTACT | |
| | GGGGCTGGATCCGCCAGCCCCCCGGGAAGGGGCTA | |
| | GAATGGATTGGGAATATCTATTATAGTGGGAGCGC | |
| | CTTCTACAACCCGTCCCTCAAGAGTCGAGTCACCA | |
| | TATCCGTAGACACGTCCAAGAACCAGTTCTCCCTG | |
| | AAGCTGAGCTCTGTGACCGCCGCAGACACGGCTGT | |
| | GTATTACTGTGCGAGATGGGATAACTGGGACGTAG | |
| | GAGCTTTTGATATCTGGGGCCAAGGGACAATGGTC | |
| | ACCGTCTCTTCA | |
| BVO-B-1H7-E3_VL | CAGTCTGCCCTGACTCAGCCTCCCTCCGCGTCCGGG | 17 |
| | TCTCCTGGACAGTCAGTCACCATCTCCTGCACTGGA | |
| | ACCAGCAGTGACGTTGGTGGTTATAACTATGTCTC | |
| | CTGGTACCAACAGCACCCAGGCAAAGCCCCCAAAC | |
| | TCATGATTTATGAGGTCAGTAAGCGGCCCTCAGGG | |
| | GTCCCTGATCGCTTCTCTGGCTCCAAGTCTGGCAAC | |
| | ACGGCCTCCCTGACCGTCTCTGGGCTCCAGGCTGA | |
| | GGATGAGGCTGATTATTACTGCACCTCATTTGCAG | |
| | GCAGCAACAATTGGGTGTTCGGCGGAGGGACCAA | |
| | GCTGACCGTCCTA | |
| BVO-B-5D2-A9_VH | CAGGTGCAGCTGCAGGAGTCGGGCCCAGGACTGGT | 18 |
| | GAAGCCTTCACAGACCCTGTCCCTCACCTGCACTG | |
| | TCTCTGGTGGCTCCATCAGCAGTAGTGGTCACTAC | |
| | TGGAGCTGGATCCGCCAGCACCCAGGGAAGGGCCT | |
| | GGAGTGGATTGGGTACATCTATTACAGTGGGGACA | |
| | CCTACTACAACCCGTCCCTCAAGAGTCGAATTACC | |
| | ATGTCAGTAGACACGTCTAAGAACCAGTTCTCCCT | |
| | GAAGCTGAGCTCTGTGACTGCCGCGGACACGGCCG | |
| | TATATTACTGTGCGAGACCGAAGTTAGATGACTGG | |
| | GCCCTTGACTACTGGGGCCAGGGAACCCTGGTCAC | |
| | CGTCTCCTCA | |
| BVO-B-5D2-A9_VL | TCCTATGTGCTGACTCAGCCACCCTCGGTGTCAGTG | 19 |
| | GCCCCAGGACAGACGGCCAGGATTACCTGTGGGGG | |
| | AAATAACATTGGAAGTAAAACTGTGCACTGGTACC | |
| | AGCAGAAGCCAGGCCAGGCCCCTGTGCTGGTCGTC | |
| | TATAATGATGGCGACCGGCCCTCAGGGATCCCTGA | |
| | GCGATTCTCTGGCTCCAACTCTGGGAACACGGCCA | |
| | CCCTGACCATCAGCAGGGTCGAAGCCGGGGATGAG | |
| | GCCGACTACTACTGTCAGGTGTGGGATAGTAGTAG | |
| | TGCTCGTGTGGTGTTCGGCGGAGGGACCAAGCTGA | |
| | CCGTCCTA | |
| BVN-B-2F11-E4_VH | GAGGTGCAGCTGTTGGAGTCTGGGGGAGGCTTGGT | 20 |
| (Control) | ACAGCCTGGGGGGTCCCTGAGACTCTCCTGTGCAG | |
| | CCTCTGGATTCACCTTTAGCACCTATGCCATGAAC | |
| | TGGGTCCGCCAGGCTCCGGGGAAGGGGCTGGAGTG | |
| | GGTCTCAGCTATTAGTGGTCGTGGTGGTAGCACAT | |
| | ACTACGCAGACTCCGTGAAGGGCCGGTTCACCATC | |
| | TCCAGAGACAATTCCAAGAACACGCTGTATCTGCA | |
| | AATGAACAGCCTGAGAGCCGAGGACACGGCCGTA | |
| | TATTACTGTGCGAAAAGGGGGCTGGGGGGTCCATT | |
| | TGACTACTGGGGCCAGGGAACCCTGGTCACCGTCT | |
| | CCTCA | |
| BVN-B-2F11-E4-VL | GACATCCAGATGACCCAGTCTCCATCCTCACTGTCT | 21 |
| (Control) | GCATCTGTAGGAGACAGAGTCACCATCACTTGTCG | |
| | GGCGAGTCAGGGCATTAGCAATTATTTAGCCTGGT | |
| | TTCAGCAGAAACCAGGGAAAGCCCCTAAGTCCCTG | |
| | ATCTATGGTGCATCCAGTTTGCAAAGTGGGGTCCC | |
| | ATCAAAGTTCAGCGGCAGTGGATCTGGGACAGATT | |
| | TCACTCTCACCATCAGCAGCCTGCAGCCTGAAGAT | |
| | TTTGCAACTTATTACTGCCAACAGTATAATAGTTA | |
| | CCCTCGGACGTTCGGCCAAGGGACCAAGGTGGAAA | |
| | TCAAA | |
| BVN-A-2C7-G4_VH | GAGGTCCAGCTGTTGGAGTCTGGGGGAGGCTTGGT | 22 |
| | CCAGCCTGGGGGGTCCCTGAGACTCTCCTGTGCAG | |
| | CCTCTGGATTCGCCTTTAACAGCTTTGCCATGAGT | |
| | TGGGTCCGCCAGGCTCCAGGGAAGGGGCTGGACTG | |
| | GGTCTCAGCTATTGGTGGTCGTGGTGGTAGCACAT | |
| | ACTACGCAGACTCCGTGAAGGGCCGGTTCACCATC | |
| | TCCCGAGACAATTCCAAGAACACGCTGTTTCTGCA | |
| | AATGAACAGCCTGAGAGCCGAGGACACGGCCGTAT | |
| | ATTACTGTGCGAGCCAACCCACGCCCCCCTTTGAC | |
| | TACTGGGGCCAGGGAACCCTGGTCACCGTCTCCTC | |
| | A | |
| BVN-A-2C7-G4_VL | GCCATCCAGATGACCCAGTCTCCATCCTCCCTGTCT | 23 |
| | GCATCTGTAGGAGACAGAGTCACCATCACTTGCCG | |
| | GGCAAGTCAGGACATTAGAAATGATTTAGGCTGGT | |
| | ATCAGCAGAGACCAGGGAAAGCCCCTCAACTCCTG | |
| | ATCTATGCTACATCCATTTTACAAAGTGGGGTCCC | |
| | ATCAAGGTTCAGCGGCAGTGGATCTGGCACAGTTT | |
| | TCACTCTCACCATCAGCAGCCTGCAGCCTGAAGAT | |
| | TTTGCGACTTATTACTGTCTACAAGATTACAATTA | |
| | CCCGTGCAATTTTGGCCAGGGGACCAAGCTGGAGA | |
| | TCAAA | |
| BVN-B-5B5-A4_VH | CAGGTGCAGCTGGTGCAGTCTGGGGCTGAGGTGAA | 24 |
| | GAAGCCTGGGGCCTCAGTGAAGGTCTCCTGCAAGG | |
| | CTTCTGGATACACCTTCACCGGCTACTATATCCACT | |
| | GGGTGCGACAGGCCCCTGGACAAGGGCTTGAGTGG | |
| | ATGGGACGGATCGTCCCTAAAAGTGGTGTCACAAA | |
| | CTATGCACAGAAGTTTCAGGGCAGGGTCACCATGA | |
| | CCAGGGACACGTCCATCAGCGCAGCCTACATGGAG | |
| | CTGAGCAGGCTGAGATCTGACGACACGGCCATGTA | |
| | TTACTGTGCGAGAGACGGTCAGTGGCTGCCTGACT | |
| | ACTGGAGCCAGGGAACCCTGGTCACCGTCTCCTCA | |
| BVN-B-5B5-A4_VL | GACATCCAGATGACCCAGTCTCCATCCTCCCTGTCT | 25 |
| | GCATCTGTAGGAGACAGAGTCACCATCACTTGCCG | |
| | GGCAAGTCAGGGCATTAGAAATGATTTAGGCTGGT | |
| | ATCAACAGAAACCAGGGAAAGCCCCTAAGCGCCT | |
| | GATCTATGCTGCATCCAGTTTGCACAGTGGGGTCC | |
| | CATCAAGGTTCAGCGGCAGTGGATCTGGGACAGAA | |
| | TTCACTCTCACAATCAGCAGCCTGCAGCCTGAAGA | |
| | TTTTGCAACTTATTACTGTCTACAGCATAATAGTTA | |
| | CCCGTACACTTTTGGCCAGGGGACCAGGCTGGACA | |
| | TCAAA | |

The present disclosure provides antibodies comprising the same CDR sequences and/or the same framework region sequences and/or the same variable region sequences as one or more of the sequences disclosed in Table 2. For example, in some embodiments, the disclosed anti-ALK1 antibody may comprise a heavy chain variable region comprising the CDRs of SEQ ID NO: 2 and a light chain variable region comprising the CDRs of SEQ ID NO: 3. In some embodiments, the disclosed anti-ALK1 antibody may comprise a heavy chain variable region comprising the CDRs of SEQ ID NO: 4 and a light chain variable region comprising the CDRs of SEQ ID NO: 5. In some embodiments, the disclosed anti-ALK1 antibody may comprise a heavy chain variable region comprising the CDRs of SEQ ID NO: 6 and a light chain variable region comprising the CDRs of SEQ ID NO: 7. In some embodiments, the disclosed anti-ALK1 antibody may comprise a heavy chain variable region comprising the CDRs of SEQ ID NO: 10 and a light chain variable region comprising the CDRs of SEQ ID NO: 11. In some embodiments, the disclosed anti-ALK1 antibody may comprise a heavy chain variable region comprising the CDRs of SEQ ID NO: 12 and a light chain variable region comprising the CDRs of SEQ ID NO: 13.

In some embodiments, the anti-ALK1 antibody may comprise a heavy chain comprising a CDRH1 comprising SSYWN (SEQ ID NO: 26), a CDRH2 comprising EVNHSGSTNYNPSLKS (SEQ ID NO: 27), and a CDRH3 comprising SPRSGRIVGAVFDY (SEQ ID NO: 28); and a light chain comprising a CDRL1 comprising GGNNIGPKSVH (SEQ ID NO: 29), a CDRL2 comprising DDSDRPS (SEQ ID NO: 30), and a CDRL2 comprising QVWDSSNDHVV (SEQ ID NO: 31). In some embodiments, the anti-ALK1 antibody is BVO-A-9F7-A10.

In some embodiments, the anti-ALK1 antibody may comprise a heavy chain comprising a CDRH1 comprising SRSYYWG (SEQ ID NO: 32), a CDRH2 comprising NIYYSGSAFYNPSLKS (SEQ ID NO: 33), and a CDRH3 comprising WDNWDVGAFDI (SEQ ID NO: 34); and a light chain comprising a CDRL1 comprising TGTSSDVGGYNYVS (SEQ ID NO: 35), a CDRL2 comprising EVSKRPS (SEQ ID NO: 36), and a CDRL2 comprising TSFAGSNNWV (SEQ ID NO: 37). In some embodiments, the anti-ALK1 antibody is BVO-B-1H7-E3.

In some embodiments, an anti-ALK1 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 2, 4, 6, 10, or 12. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, but an anti-ALK1 antibody comprising that sequence retains the ability to bind to ALK1. In some embodiments, a total of 1 to 10 amino acids (e.g., 1, 2, 3, 4, 5, 6, 7, 8, 9, or 10 amino acids) have been substituted, inserted and/or deleted in SEQ ID NO: 2, 4, 6, 10, or 12. In some embodiments, substitutions, insertions, or deletions occur in regions outside the CDRs (that is, in the FRs). Optionally, the anti-ALK1 antibody comprises the VH sequence in SEQ ID NO: 2, 4, 6, 10, or 12, including post-translational modifications of that sequence.

In some embodiments, an anti-ALK1 antibody comprises a heavy light variable domain (VL) sequence having at least 90%, 91%, 92%, 93%, 94%, 95%, 96%, 97%, 98%, 99%, or 100% sequence identity to the amino acid sequence of SEQ ID NO: 3, 5, 7, 11, or 13. In some embodiments, a VL sequence having at least 90%, 91%, 92%, 93%, 94%, 95%, 96%, 97%, 98%, or 99% identity contains substitutions (for example, conservative substitutions), insertions, or deletions relative to the reference sequence, but an anti-ALK1 antibody comprising that sequence retains the ability to bind to ALK1. In some embodiments, a total of 1 to 10 amino acids (e.g., 1, 2, 3, 4, 5, 6, 7, 8, 9, or 10 amino acids) have been substituted, inserted and/or deleted in SEQ ID NO: 3, 5, 7, 11, or 13. In some embodiments, substitutions, insertions, or deletions occur in regions outside the CDRs (that is, in the FRs). Optionally, the anti-ALK1 antibody comprises the VL sequence in SEQ ID NO: 3, 5, 7, 11, or 13, including post-translational modifications of that sequence.

As noted above, the disclosed antibodies can bind to various mammalian ALK1 proteins, including the human, murine, and cynomolgus. For example, BVO-B-1H7-E3 binds to human, murine, and cynomolgus ALK1 and BVO-A-9E7-A10 binds to human and cynomolgus ALK1. In contrast, BVN-B-2F11-E4 (control) only shows a weak binding to human ALK1.

In addition to binding ALK1, the disclosed antibodies can possess other beneficial properties as well. For example, the disclosed antibodies may not block BMP9 (Bone Morphogenetic Protein-9) signaling and allow for phosphorylation of SMAD 1/5. BMP9 plays a role in inducing and maintaining the ability of embryonic basal forebrain cholinergic neurons (BFCN) to respond to a neurotransmitter called acetylcholine, and it induces the differentiation of mesenchymal stem cells (MSCs) to an osteoblast lineage via the SMAD signaling pathway. Of note for the purposes of the present disclosure, BMP9 is a ligand of ALK1. Endoglin, a type I membrane glycoprotein that forms the TGF-β receptor complex, is a co-receptor of ALK1 for GDF2/BMP-9 binding. Accordingly, the ability of certain disclosed antibodies to bind ALK1 without blocking BMP9 signaling is unique. In some embodiments, the disclosed antibodies bind to ALK1 and do not block BMP9 signaling.

Another feature of certain disclosed antibodies is the ability to bind ALK1 with little or no cross reactivity with ALK5. Like ALK1, ALK5 is a TGF-β receptor, and both receptors share similar structures and sequences. Accordingly, selective binding to ALK1 without cross reactivity with ALK5 is a desirable feature of the disclosed antibodies, as it minimizes the likelihood of off-target effects of the antibodies. In some embodiments, the disclosed antibodies do not bind to or cross react with ALK5. Neither BVO-B-1H7-E3 nor BVO-A-9E7-A10 bind to ALK5.

Any of the antibodies disclosed herein can be used for treating and/or preventing diseases and disorders associated with ALK1, such as atherosclerosis. Optimal doses and routes of administration may vary, such as based on the route of administration and dosage form, the age and weight of the subject, and/or the subject's condition, including the type and severity of the disease, and can be determined by the skilled practitioner. The antibodies can be formulated in a pharmaceutical composition suitable for administration to a subject by any intended route of administration, as discussed in more detail below.

### IV. Pharmaceutical Compositions

Provided herein are pharmaceutical compositions for use in the treatment or prevention of disease and disorders associated with ALK1, including atherosclerosis. The pharmaceutical compositions comprise an anti-ALK1 antibody described herein as an active ingredient.

Pharmaceutical compositions of an anti-ALK1 antibody of the present disclosure can be prepared as formulations according to standard methods (see, for example, Remington's Pharmaceutical Science, Mark Publishing Company, Easton, USA). The pharmaceutical compositions generally comprise a carrier and/or additive in addition to the antibody. For example, in some embodiments, the pharmaceutical composition comprises one or more surfactants (for example, PEG and Tween), excipients, antioxidants (for example, ascorbic acid), coloring agents, flavoring agents, preservatives, stabilizers, buffering agents (for example, phosphoric acid, citric acid, and other organic acids), chelating agents (for example, EDTA), suspending agents, isotonizing agents, binders, disintegrators, lubricants, fluidity promoters, corrigents, light anhydrous silicic acid, lactose, crystalline cellulose, mannitol, starch, carmelose calcium, carmelose sodium, hydroxypropylcellulose, hydroxypropylmethylcellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, and inorganic salt. In some embodiments, the pharmaceutical composition comprises one or more other low-molecular-weight polypeptides, proteins such as serum albumin, gelatin, and immunoglobulin, and amino acids such as glycine, glutamine, asparagine, arginine, and lysine.

An anti-ALK1 antibody may be prepared as an aqueous solution for injection, in which the anti-ALK1 antibody can be dissolved in an isotonic solution containing, for example, physiological saline, dextrose, or other excipients or tonifiers (i.e., tonicity agents). The tonifier may include, for example, D-sorbitol, D-mannose, D-mannitol, and sodium chloride. In addition, appropriate solubilizing agents, for example, alcohols (for example, ethanol), polyalcohols (for example, propylene glycols and PEGs), and non-ionic detergents (polysorbate 80 and HCO-50) may be used concomitantly.

The disclosed pharmaceutical compositions may be formulated for any suitable route of administration, including intravenous, subcutaneous, intraperitoneal, intramuscular, or oral administration. In typical embodiments, the anti-ALK1 antibodies are formulated for intravenous, subcutaneous, intraperitoneal, or intramuscular administration, such as in a solution, suspension, emulsion, liposome formulation, etc. More specifically, the disclosed anti-ALK1 antibodies can be formulated for intravenous, subcutaneous, or intramuscular administration.

Pharmaceutically acceptable carriers for various dosage forms and routes of administration are known in the art. For example, solvents, solubilizing agents, suspending agents, isotonicity agents, buffers, and soothing agents for liquid preparations are known. In some embodiments, the pharmaceutical compositions include one or more additional components, such as one or more preservatives, antioxidants, colorants, sweetening/flavoring agents, adsorbing agents, wetting agents and the like.

The present disclosure provides a pharmaceutical composition for use in the treatment or prevention of diseases and disorders associated with ALK1, such as atherosclerosis, in a subject, wherein the pharmaceutical composition comprises an anti-ALK1 antibody disclosed herein as an active ingredient

Any of the pharmaceutical compositions disclosed herein can be used for treating and/or preventing diseases and disorders associated with ALK1, such as atherosclerosis. Optimal doses and routes of administration may vary.

### V. Treatments and Preventions

The present disclosure provides methods of treating or preventing diseases and disorders associated with ALK1, such as atherosclerosis. Additionally, the present disclosure provides methods of blocking an interaction between ALK1 and a low density lipoprotein (LDL). Additionally, the present disclosure provides methods of blocking ALK1 binding to LDL. Additionally, the present disclosure provides uses of the disclosed anti-ALK1 antibodies for treating or preventing diseases and disorders associated with ALK1, such as atherosclerosis. Additionally, the present disclosure provides use of the disclosed anti-ALK1 antibodies for blocking an interaction between ALK1 and a low density lipoprotein (LDL). Additionally, the present disclosure provides use of the disclosed anti-ALK1 antibodies for blocking ALK1 binding to LDL

As noted above, ALK1 can play an important role in the buildup of LDL cholesterol in blood vessels. The disclosed antibodies (e.g., BVO-A-9F7-A10 and BVO-B-1H7-E3) are able to block LDL accumulation by disrupting the “LDL-ALK1 pathway” in the walls of blood vessels that facilitates the transport of LDL from blood into tissue (aka LDL transcytosis). This in turn can help prevent or slow the clogging of arteries that leads to heart disease. The conformation selective ALK1 binding monoclonal antibodies disclosed herein (e.g., BVO-A-9F7-A10 and BVO-B-1H7-E3) efficiently block LDL transcytosis into the endothelium, but not BMP9 signaling, resulting in a dramatic reduction in plaque formation. Thus, the disclosed antibodies are particularly well suited for the treatment and/or prevention of atherosclerosis.

Atherosclerosis is the buildup of fats, cholesterol and other substances in and on your artery walls. This buildup is called plaque. The plaque can cause your arteries to narrow, blocking blood flow. The plaque can also burst, leading to a blood clot. Although atherosclerosis is often considered a heart problem, it can affect arteries anywhere in your body. Mild atherosclerosis usually does not have any symptoms, but moderate to severe atherosclerosis may cause chest pain or pressure (angina), high blood pressure, and kidney failure, depending on the location of plaque formation. Atherosclerosis can also lead to the development of co-morbidities, such as transient ischemic attack (TIA), stroke, peripheral artery disease, and heart failure.

In general, the treatments disclosed herein comprise administering to a subject a therapeutically effective amount of an anti-ALK1 antibody (e.g., BVO-A-9F7-A10 or BVO-B-1H7-E3). In particular, the present disclosure provides methods of treating or preventing atherosclerosis, comprising administering to a subject with atherosclerosis an anti-ALK1 antibody (e.g., BVO-A-9F7-A10 or BVO-B-1H7-E3) disclosed herein. In some embodiments, the antibody may reduce the risk of developing atherosclerosis in a subject that has not yet developed plaques. In some embodiments, the antibody may inhibit or reduce further plaque formation. In some embodiments, the antibody may break up existing plaques or reduce the size or number of existing plaques.

An effective amount of a disclosed anti-ALK1 antibody can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the anti-ALK1 antibodies of the present disclosure for any particular subject may depend upon a variety of factors including the activity of the specific compound employed, the age, body weight, general health, sex, and diet of the subject, the time of administration, the rate of excretion, the drug combination, and the severity of the particular disorder being treated and form of administration. Treatment and prevention dosages generally may be titrated to optimize safety and efficacy. The dosage can be determined by a physician and adjusted, as necessary, to suit observed effects of the treatment. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro.

Administration of the anti-ALK1 antibody (e.g., BVO-A-9F7-A10 or BVO-B-1H7-E3) may be intravenous, subcutaneous, intramuscular, intradermal, or intraperitoneal. In general, injections or infusions of the antibody will be the most common route of administration. Administration may comprise a single dose or repeated dosing over a period of treatment.

In some embodiments of the disclosed methods and uses, an anti-ALK1 antibody is administered daily, every other day, twice per week, three times per week, four times per week, five times per week, six times per week, once per week, once every two weeks, once every three weeks, once every four weeks, once every five weeks, once every six weeks, once every seven weeks, once every eight weeks, once every nine weeks, once every 10 weeks, once every 11 weeks, once every 12 weeks, twice per year, once per year, and/or as needed.

In some embodiments of the disclosed methods and uses, the duration of treatment or prevention is about one day, about one week, about two weeks, about three weeks, about four weeks, about five weeks, about six weeks, about seven weeks, about eight weeks, about nine weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 24 weeks, about 30 weeks, about 36 weeks, about 40 weeks, about 48 weeks, about 50 weeks, about one year, about two years, about three years, about four years, about five years, or as needed based.

The present disclosure provides uses of an anti-ALK1 antibody in the manufacture of a medicament for the treatment or prevention of diseases and disorders associated with ALK1, such as atherosclerosis.

### EXAMPLES

The examples discussed below are intended to be purely exemplary of the invention and should not be considered to limit the invention in any way. The examples are not intended to represent that the experiments below are all or the only experiments performed.

### Example 1—Isolation of ALK1-Binding Antibodies

OmniRats were immunized using the Gene Gun method, i.e. gold particles were loaded with DNA and administered into the skin of the animals using a Gene Gun (Helios). In other words, ALK1-transfected/expressing cells were not injected for immunization, but plasmid DNA was used directly, which induced expression of ALK1 in the skin cells/DCs of Omni rats.

Polyclonal serum of immunized animals were tested for activity. B-cells of interest were isolated, and hyridomas were made. Following isolations and subcloning of the hybridomas, the supernatants were used to reassess the monoclonal antibodies.

### Example 2—ALK1 Binding Assessment

Various isolated antibodies were assessed for the ability to bind ALK1 from mouse (murine), human, and cyno monkey. Flow cytometry was used to measure fluorescence intensity produced by fluorescent-labeled secondary antibodies detecting primary anti-ALK1 antibodies that bind to specific cell-associated ALK1 molecules. More precisely hybridoma supernatants containing primary antibodies were tested for binding to transiently transfected cells expressing ALK1 of human, cyno monkey and mouse by flow cytometry. The signals were generated by use of secondary, PE-labelled goat anti-rat antibodies. In addition to testing the supernatants on non-transfected cells, ALK1 of human, cyno monkey and mouse-transfected cells were incubated only with the secondary antibody (without supernatants) as a negative control. Exemplary results are shown in the FIG. 1.

As can be seen in the FIG. 1, BVO-A-9F7-A10 and BVO-B-1H7-E3 showed strong binding to human and cyno ALK1, but less cross-reactivity to the murine orthologue.

It should be noted that the expression of the murine ALK1 seems to be much weaker when compared to the cyno and human ALKs. Moreover, as this is done via binding of an anti-tag antibody to a N-terminal tag, it was often found that the signal obtained was not necessarily reflecting the “real” cell surface expression level of a certain protein. Often the “real” expression was higher than indicated by the signal obtained by the tag, possibly due to the fact that the tag sequence might not be fully accessible for the anti-tag ab, resulting in a lower signal. For example, it is known that this phenomenon is dependent even on the type of tag (myc, FLAG or His etc.) used—with the same antigen, some suggest a lower/higher expression level then others—which cannot be confirmed in a parallel test with a specific (anti-target) antibody. As shown in the FIG. 1, the specific anti-ALK1 antibodies give much higher signals than the expression control generated with an anti-tag antibody.

All that said, it cannot deduced from the low signals on the murine ALK1 that the abs in question have only a very low anti-murine cross-reactivity. It may be that the low signal comes from a much low(er) cell surface expression of the murine ALK1 when compared to cyno and human, indicated by the much lower expression control.

In any event, the disclosed antibodies, and BVO-A-9F7-A10 and BVO-B-1H7-E3 in particular, showed strong and specific binding affinity for multiple mammalian ALK1 orthologs.

### Example 3—ALK5 Binding Assessment

Flow cytometry was used to measure fluorescence intensity produced by fluorescent-labeled secondary antibodies detecting primary anti-ALK1 antibodies that bind to specific cell-associated ALK5 molecules. More precisely hybridoma supernatants containing primary antibodies were tested for binding to transiently transfected cells expressing ALK5 of human and mouse by flow cytometry. The signals were generated by use of secondary, PE-labelled goat anti-rat antibodies. In addition to testing the supernatants on non-transfected cells, ALK5 of human and mouse-transfected cells were incubated only with the secondary antibody (without supernatants) as a negative control.

### Example 4—BMP9 Signaling Assessment

Eighty-nine (89) anti-ALK1 antibodies were assessed to determine whether the antibodies inhibited BMP9 binding and SMAD1/5 signaling. Of the 89 antibodies that were assessed, a total of 12 did not block BMP9 signaling.

TABLE 4
Antibodies Screened for BMP9 Signaling Inhibition

| No. | Antibody Name |
|---|---|
| 1 | BVN-A-1A8 |
| 2 | BVN-A-1B9 |
| 3 | BVN-A-1C8 |
| 4 | BVN-A-1D9 |
| 5 | BVN-A-1E9 |
| 6 | BVN-A-1F4 |
| 7 | BVN-A-1G4 |
| 8 | BVN-A-1H3 |
| 9 | BVN-A-2B2 |
| 10 | BVN-A-2B9 |
| 11 | BVN-A-2C7 |
| 12 | BVN-A-2D10 |
| 13 | BVN-A-2E1 |
| 14 | BVN-A-2E11 |
| 15 | BVN-A-2H2 |
| 16 | BVN-A-2H3 |
| 17 | BVN-A-2H5 |
| 18 | BVN-B-1B7 |
| 19 | BVN-B-1C11 |
| 20 | BVN-B-1E2 |
| 21 | BVN-B-2A7 |
| 22 | BVN-B-2E2 |
| 23 | BVN-B-2F9 |
| 24 | BVN-B-2F11 |
| 25 | BVN-B-3B11 |
| 26 | BVN-B-3E11 |
| 27 | BVN-B-4A5 |
| 28 | BVN-B-4B7 |
| 29 | BVN-B-4C4 |
| 30 | BVN-B-4C9 |
| 31 | BVN-B-4E9 |
| 32 | BVN-B-5B5 |
| 33 | BVN-B-5B11 |
| 34 | BVN-B-5D9 |
| 35 | BVN-B-5F11 |
| 36 | BVO-A-1D1 |
| 37 | BVO-A-1D5 |
| 38 | BVO-A-1D11 |
| 39 | BVO-A-2C11 |
| 40 | BVO-A-3F6 |
| 41 | BVO-A-3G10 |
| 42 | BVO-A-4A11 |
| 43 | BVO-A-4B5 |
| 44 | BVO-A-4B9 |
| 45 | BVO-A-6A10 |
| 46 | BVO-A-6H9 |
| 47 | BVO-A-7C11 |
| 48 | BVO-A-7D1 |
| 49 | BVO-A-7G10 |
| 50 | BVO-A-8C11 |
| 51 | BVO-A-8E9 |
| 52 | BVO-A-8F8 |
| 53 | BVO-A-9C4 |
| 54 | BVO-A-9C5 |
| 55 | BVO-A-9F7 |
| 56 | BVO-A-11E5 |
| 57 | BVO-A-12B7 |
| 58 | BVO-A-12E2 |
| 59 | BVO-A-12E10 |
| 60 | BVO-A-12F6 |
| 61 | BVO-A-12G10 |
| 62 | BVO-A-13B7 |
| 63 | BVO-A-13C8 |
| 64 | BVO-B-1F4 |
| 65 | BVO-B-1G4 |
| 66 | BVO-B-1H7 |
| 67 | BVO-B-3C7 |
| 68 | BVO-B-4G2 |
| 69 | BVO-B-5D2 |
| 70 | BVO-B-6B2 |
| 71 | BVO-B-6H7 |
| 72 | BVO-B-9E1 |
| 73 | BVO-B-9G7 |
| 74 | BWG-2E5 |
| 75 | BWG-3B3 |
| 76 | BWG-3B7 |
| 77 | BWG-3F4 |
| 78 | BWG-5C10 |
| 79 | BWG-5E8 |
| 80 | BWG-6C4 |
| 81 | BWG-6C10 |
| 82 | BWG-6D11 |
| 83 | BWG-7C11 |
| 84 | BWG-7F2 |
| 85 | BWG-8E11 |
| 86 | BWG-9C5 |
| 87 | BWG-10E6 |
| 88 | BWG-10F11 |
| 89 | BWF-4G2 |
| — | — |

The following table shows the antibodies that were assessed via the above-described assay. Of these antibodies, BVN-A-2B2, BVN-A-2C7, BVN-A-2D10, BVN-B-2F11, BVN-B-5B5, BVO-A-1D5, BVO-A-9C4, BVO-A-9C5, BVO-A-9F7, BVO-B-1H7, BVO-B-5D2, and BWG-3B3 did not block BMP9 signaling.

### Example 5—Atherosclerosis Experiment

BVO-A-9F7-A10 has been shown to block LDL uptake, but not BMP9 signaling, in cultured human and mouse endothelial cells. In addition, in a preliminary experiment, treatment of mice with anti-ALK antibody overnight reduces LDL uptake into the vessel wall in vivo. BVO-B-1H7-E3 is expected to produce similar results.

The goal of the follow prophetic study is to examine if chronic treatment with ALK1 antibody reduces atherosclerosis in a mouse model.

### Experimental Plan

8-10 week old mice lacking the LDL receptor are fed a cholesterol rich high fat diet (HFD) for 14 weeks to induce experimental atherosclerosis.

Mice are treated with control or anti-ALK Ab (e.g., BVO-A-9F7-A10 or BVO-B-1H7-E3; 400-500 micrograms per mouse x 3 times per week) for 14 weeks duration.

At 14 weeks, the following analysis will be performed:
1. Plasma lipids (cholesterol, TG) and inflammatory cell populations by FACs
2. Plasma collection for levels of Ab (PK assessment)
3. Assessment of the extent of atherosclerosis in 3 anatomical locations: 1. the aorta (en face imaging of neutral lipid accumulation), 2. Cross sections of the aortic roots and 3. The brachiocephalic arteries.
4. Analysis of lesion composition including ApoB 100 immunochemistry, macrophages (F4/80 or CD68 staining), lesion necrosis and fibrous caps (from H&E sections).

The results will indicate that the disclosed antibodies (e.g., BVO-A-9F7-A10 and BVO-B-1H7-E3) are suitable for treating or preventing atherosclerosis.

All patents and publications mentioned in the specification are indicative of the levels of those of ordinary skill in the art to which the disclosure pertains. All patents and publications are herein incorporated by reference to the same extent as if each individual publication was specifically and individually indicated to be incorporated by reference.

Further, one skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

1. An anti-ALK1 antibody, comprising the complementarity determining regions (CDRs) of a heavy chain variable sequence and a light chain variable sequence selected from:
a. the heavy chain variable sequence of SEQ ID NO: 2 and the light chain variable sequence of SEQ ID NO: 3;
b. the heavy chain variable sequence of SEQ ID NO: 4 and the light chain variable sequence of SEQ ID NO: 5;
c. the heavy chain variable sequence of SEQ ID NO: 6 and the light chain variable sequence of SEQ ID NO: 7;
d. the heavy chain variable sequence of SEQ ID NO: 10 and the light chain variable sequence of SEQ ID NO: 11; and
e. the heavy chain variable sequence of SEQ ID NO: 12 and the light chain variable sequence of SEQ ID NO: 13.

2. The anti-ALK1-antibody of claim 1, further comprising a variable heavy chain sequence and a variable light chain sequence selected from:
a. the heavy chain variable sequence of SEQ ID NO: 2 and the light chain variable sequence of SEQ ID NO: 3;
b. the heavy chain variable sequence of SEQ ID NO: 4 and the light chain variable sequence of SEQ ID NO: 5;
c. the heavy chain variable sequence of SEQ ID NO: 6 and the light chain variable sequence of SEQ ID NO: 7;
d. the heavy chain variable sequence of SEQ ID NO: 10 and the light chain variable sequence of SEQ ID NO: 11; and
e. the heavy chain variable sequence of SEQ ID NO: 12 and the light chain variable sequence of SEQ ID NO: 13.

3. The anti-ALK1 antibody of claim 1, wherein the heavy chain variable sequence comprises at least 90%, 91%, 92%, 93%, 94%, 95%, 96%, 97%, 98%, 99%, or 100% sequence identity to SEQ ID NO: 2, 4, 6, 10, or 12, and the light chain variable sequence comprises at least 90%, 91%, 92%, 93%, 94%, 95%, 96%, 97%, 98%, 99%, or 100% sequence identity to SEQ ID NO: 3, 5, 7, 11, or 13.

4. The anti-ALK1 antibody of claim 1, wherein the heavy chain variable sequence comprises 1-10 amino acid substitutions, insertions or deletions in SEQ ID NO: 2, 4, 6, 10, or 12, and the light chain variable sequence comprises 1-10 amino acid substitutions, insertions or deletions in SEQ ID NO: 3, 5, 7, 11, or 13.

5. An anti-ALK1 antibody comprising a heavy chain comprising a CDRH1 comprising SSYWN (SEQ ID NO: 26), a CDRH2 comprising EVNHSGSTNYNPSLKS (SEQ ID NO: 27), and a CDRH3 comprising SPRSGRIVGAVFDY (SEQ ID NO: 28); and a light chain comprising a CDRL1 comprising GGNNIGPKSVH (SEQ ID NO: 29), a CDRL2 comprising DDSDRPS (SEQ ID NO: 30), and a CDRL2 comprising QVWDSSNDHVV (SEQ ID NO: 31).

6. An anti-ALK1 antibody comprising a heavy chain comprising a CDRH1 comprising SRSYYWG (SEQ ID NO: 32), a CDRH2 comprising NIYYSGSAFYNPSLKS (SEQ ID NO: 33), and a CDRH3 comprising WDNWDVGAFDI (SEQ ID NO: 34); and a light chain comprising a CDRL1 comprising TGTSSDVGGYNYVS (SEQ ID NO: 35), a CDRL2 comprising EVSKRPS (SEQ ID NO: 36), and a CDRL2 comprising TSFAGSNNWV (SEQ ID NO: 37).

7. A method of treating or preventing a disease or disorder associated with ALK1, comprising administering to a subject with a disease or disorder associated with ALK1 an antibody of any one of claims 1-6.

8. The method of claim 7, wherein the disease is atherosclerosis.

9. The method of claim 7 or 8, wherein the subject is human.
